(19) United States
(12) Patent Application Publication
(10) Pub. No.: US 2026/0060638 A1
(43) Pub. Date: Mar. 5, 2026

(54) ULTRASONIC DIAGNOSTIC APPARATUS, CONTROL METHOD OF ULTRASONIC DIAGNOSTIC APPARATUS, AND NON-TRANSITORY COMPUTER READABLE MEDIUM

(71) Applicants: CANON KABUSHIKI KAISHA, Tokyo (JP); CANON MEDICAL SYSTEMS CORPORATION, Tochigi (JP)

(72) Inventors: NAOYA IIZUKA, Kanagawa (JP); SHOYA SASAKI, Tokyo (JP); TAKESHI SATO, Tochigi (JP); HIROKI TAKAHASHI, Tochigi (JP)

(21) Appl. No.: 19/316,141

(22) Filed: Sep. 2, 2025

(30) Foreign Application Priority Data
Sep. 5, 2024 (JP) 2024-153377

(51) Int. Cl. A61B 8/06; A61B 8/00

(52) U.S. Cl. CPC A61B 8/06; A61B 8/488; A61B 8/5207; A61B 8/5246

(57) ABSTRACT

An ultrasonic diagnostic apparatus executes transmitting and receiving ultrasonic waves with respect to a subject, executes acquiring data of reflected waves in the subject, executes generating a first blood flow image indicating a blood flow of the subject, executes generating a second blood flow image including a component with a lower spatial frequency than the first blood flow image, executes detecting maximum values of blood flow signals in a predetermined range of the second blood flow image, executes calculating a blood flow velocity and a blood flow direction of the subject based on positional information of the maximum values, and executes generating a third blood flow image based on the first blood flow image, the blood flow velocity, and the blood flow direction.

### BACKGROUND

### Field of the Technology

The present disclosure relates to an ultrasonic diagnostic apparatus, a control method of an ultrasonic diagnostic apparatus, and a non-transitory computer readable medium.

### Description of the Related Art

Ultrasonic diagnostic apparatuses are widely used to observe and diagnose blood flow in living organisms. Ultrasonic diagnostic apparatuses generate blood flow information of a living organism using reflected ultrasonic waves from the living organism by the Doppler method based on the Doppler effect. Blood flow information generated by ultrasonic diagnostic apparatuses includes color Doppler images and Doppler waveforms (Doppler spectra).

A color Doppler image is an ultrasonic image obtained by imaging using the color flow mapping (CFM) method. In the CFM method, transmission/reception of ultrasonic waves are performed a plurality of times on a plurality of scan lines. In the CFM method, by applying a moving target indicator (MTI) filter with respect to a data string at the same position of a living organism, signals (clutter signals) originating from stationary tissue or slow-moving tissue are suppressed and signals originating from blood flow are extracted. In addition, in the CFM method, blood flow information such as a blood flow velocity, a blood flow dispersion, and a blood flow power is estimated from the extracted blood flow signals and a distribution of estimated results is generated as a Doppler image.

The resolution of B-mode images or Doppler images is known to decline due to the point spread function (PSF) that is determined by a wavelength of transmitted ultrasonic waves, a transmitter/receiver aperture width, and the like. While solutions to this problem include increasing the frequency of the transmitted ultrasonic waves, since there is a limit to the frequency bandwidth of ultrasonic waves that can be realized in a probe, there is also a limit to the resolution of the images that can be generated.

Super Resolution Ultrasound using Recursive Imaging of Highly Dense Scatterers, Mostafa Amin-Naji, Jorgen Arendt Jensen, Proceedings 2022 IEEE International Ultrasonics Symposium (IUS) describes a super-resolution technique for blood flow images that achieves a resolution of about ⅕ of the wavelength of the ultrasonic waves transmitted from a probe.

In Super Resolution Ultrasound using Recursive Imaging of Highly Dense Scatterers, Mostafa Amin-Naji, Jorgen Arendt Jensen, Proceedings 2022 IEEE International Ultrasonics Symposium (IUS), the resolution of generated blood flow images is improved by acquiring a large number of Doppler images and integrating the peaks of the image values of the acquired Doppler images. Furthermore, Super Resolution Ultrasound using Recursive Imaging of Highly Dense Scatterers, Mostafa Amin-Naji, Jorgen Arendt Jensen, Proceedings 2022 IEEE International Ultrasonics Symposium (IUS) proposes a technique to display a blood flow velocity and a blood flow direction by tracking peaks between consecutive frames.

In peak tracking for blood flow velocity calculation in blood flow images, the calculation of accurate blood flow velocity may not be realized due to the effect of a density of peak points and the like.

### SUMMARY

The technique according to the present disclosure has been devised in consideration thereof and an object is to provide an ultrasonic diagnostic apparatus that improves accuracy of blood flow images using more accurate information on a blood flow velocity and a blood flow direction.

According to some embodiments, an ultrasonic diagnostic apparatus includes a processor, and a memory storing a program which, when executed by the processor, causes the ultrasonic diagnostic apparatus to execute ultrasonic wave transmission and reception processing of transmitting and receiving ultrasonic waves with respect to a subject, execute data acquisition processing of acquiring data of reflected waves in the subject due to the transmission and reception of ultrasonic waves over a plurality of frames, execute first image generation processing of generating a first blood flow image indicating a blood flow of the subject using the data of the plurality of frames acquired by the data acquisition processing, execute second image generation processing of generating a second blood flow image including a component with a lower spatial frequency than the first blood flow image using the data of the plurality of frames acquired by the data acquisition processing, execute detection processing of detecting maximum values of blood flow signals in a predetermined range of the second blood flow image, execute calculation processing of calculating a blood flow velocity and a blood flow direction of the subject based on positional information of the maximum values detected by the detection processing, and execute third image generation processing of generating a third blood flow image based on the first blood flow image, the blood flow velocity, and the blood flow direction.

According to some embodiments, a control method of an ultrasonic diagnostic apparatus includes transmitting and receiving ultrasonic waves with respect to a subject, acquiring data of reflected waves in the subject due to transmission and reception of the ultrasonic waves over a plurality of frames, generating a first blood flow image indicating a blood flow of the subject using the data of the plurality of frames acquired by the acquiring of data, generating a second blood flow image including a component with a lower spatial frequency than the first blood flow image using the data of the plurality of frames acquired by the acquiring of data, detecting maximum values of blood flow signals in a predetermined range of the second blood flow image, calculating a blood flow velocity and a blood flow direction of the subject based on positional information of the maximum values detected by the detecting of a maximum values, and generating a third blood flow image based on the first blood flow image, the blood flow velocity, and the blood flow direction.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram showing an example of a configuration of an ultrasonic diagnostic apparatus.

FIG. 2 is a block diagram showing an example of a configuration of a received signal processing unit.

FIG. 3 is a flow chart of processing executed by an ultrasonic diagnostic apparatus according to a first embodiment.

FIGS. 4A and 4B are diagrams showing an example of a blood flow image according to the first embodiment.

FIGS. 5A and 5B are diagrams showing an example of an image showing local maximum values according to the first embodiment.

FIGS. 6A and 6B are diagrams showing an example of a movement between frames of local maximum values according to the first embodiment.

FIGS. 7A to 7C are diagrams showing an example of a blood flow image according to a second embodiment.

FIG. 8 is a flow chart of processing executed by an ultrasonic diagnostic apparatus according to a third embodiment.

FIG. 9 is a diagram showing an example of a frequency band used by the ultrasonic diagnostic apparatus according to the third embodiment.

### DESCRIPTION OF THE EMBODIMENTS

Hereinafter, embodiments of an ultrasonic diagnostic apparatus according to the present disclosure will be described in detail by way of example with reference to the accompanying drawings. However, it is to be understood that constituent elements described in the following embodiments are merely exemplary and that the technical scope of the present disclosure is not intended to be limited by any of the individual embodiments described below but to be defined by the scope of aspects.

### First Embodiment

An ultrasonic diagnostic apparatus according to a first embodiment will be described. In the ultrasonic diagnostic apparatus according to the present embodiment, an ultrasonic probe performs transmission/reception of ultrasonic waves to and from a subject. In addition, the ultrasonic diagnostic apparatus extracts low-frequency components from received signals based on reflected waves from the subject to generate low-frequency blood flow images, and calculates a blood flow velocity and a blood flow direction by tracking local maximum values such as power values in the low-frequency blood flow images from frame to frame. Furthermore, the ultrasonic diagnostic apparatus generates a super-resolution blood flow image in which the calculated blood flow velocity and the calculated blood flow direction are colored.

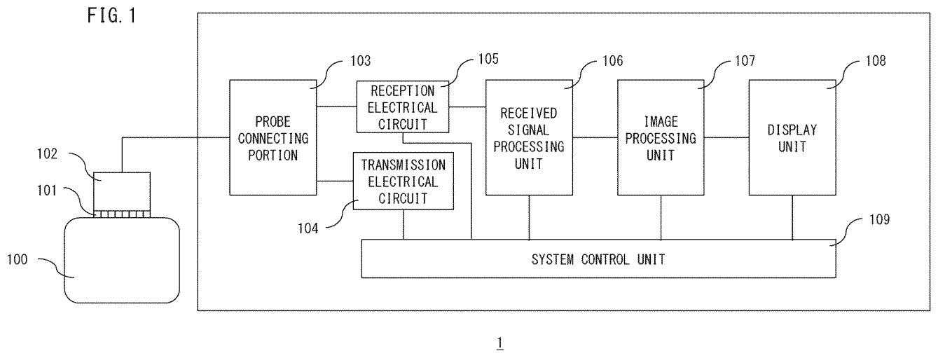

FIG. 1 is a block diagram showing an example of a hardware configuration of the ultrasonic diagnostic apparatus according to the present embodiment. An ultrasonic diagnostic apparatus 1 has an ultrasonic probe (ultrasonic transducer) 102, a probe connecting portion 103, a transmission electrical circuit 104, a reception electrical circuit 105, a received signal processing unit 106, an image processing unit 107, a display unit 108, and a system control unit 109. The ultrasonic diagnostic apparatus 1 transmits an ultrasonic pulse to a subject 100 from the ultrasonic probe 102, receives reflected ultrasonic waves having been reflected from the subject 100 with the ultrasonic probe 102, and generates image information (ultrasonic image) of the inside of the subject 100. The ultrasonic image generated by the ultrasonic diagnostic apparatus 1 is to be used in various clinical examinations.

The ultrasonic probe 102 is a probe adopting an electronic scan system and has a plurality of transducers 101 arranged one-dimensionally or two-dimensionally at a tip. The transducer 101 is an electric mechanical conversion element that performs mutual conversion between an electric signal (voltage pulse signal) and an ultrasonic wave (acoustic wave). The ultrasonic probe 102 is an ultrasonic transceiver that transmits ultrasonic waves from the plurality of transducers 101 to the subject 100 and receives reflected ultrasonic waves from the subject 100 with the plurality of transducers 101. Reflected acoustic waves from the subject 100 reflect a difference in acoustic impedances inside of the subject 100. When ultrasonic pulses transmitted from the ultrasonic probe 102 are reflected off a moving blood flow, heart wall, or another surface of the subject 100, a reflected wave signal undergoes a frequency deviation that depends on a velocity component of the moving object in an ultrasonic transmission direction due to the Doppler effect.

The transmission electrical circuit 104 outputs a pulse signal (drive signal) to the transducer 101 of the ultrasonic probe 102 via the probe connecting portion 103. The transmission electrical circuit 104 applies pulse signals to the plurality of transducers 101 with time differences. Accordingly, ultrasonic waves with different delay times are transmitted from the plurality of transducers 101 and a transmission ultrasonic beam is formed. In addition, by selectively changing the transducer 101 to which the pulse signal is applied (in other words, the transducer 101 to be driven) and changing a delay time (an application timing) of the pulse signal, the transmission electrical circuit 104 can control a direction and a focus of the transmission ultrasonic beam. An observation region inside of the subject 100 is scanned by sequentially changing the direction and the focus of the transmission ultrasonic beam.

In addition, by varying the delay time of the pulse signal, the transmission electrical circuit 104 can form a transmission ultrasonic beam that is a plane wave (focus is far away) or a diffuse wave (focus point is the opposite of the ultrasonic transmission direction with respect to the plurality of transducers 101). In a similar manner, by varying the delay time of the pulse signal, the transmission electrical circuit 104 can form a transmission ultrasonic beam using one transducer or a part of the plurality of transducers 101. By transmitting a pulse signal with a predetermined driving waveform to the transducers 101, the transmission electrical circuit 104 can generate a transmission ultrasonic wave having a predetermined transmission waveform in the transducers 101.

The reception electrical circuit 105 receives an electric signal output from the transducer 101 having received a reflected ultrasonic wave from the subject. The reception electrical circuit 105 inputs the received signal to the received signal processing unit 106. Operations of the transmission electrical circuit 104 and the reception electrical circuit 105 or, in other words, transmission/reception of ultrasonic waves is controlled by the system control unit 109. The system control unit 109 changes a timing at which a voltage signal or a transmission ultrasonic wave is formed in accordance with, for example, respective processing of generation processing of a B-mode image and generation processing of a blood flow image to be described later.

When generating a B-mode image, the ultrasonic diagnostic apparatus 1 acquires a received signal of a reflected ultrasonic wave obtained by scanning an observation region of the subject and generates a B-mode image using the acquired received signal. In addition, when generating a blood flow image, the ultrasonic diagnostic apparatus 1 acquires received signals of reflected ultrasonic waves of a plurality of frames by performing transmission/reception of an ultrasonic wave a plurality of times on one scan line or each of a plurality of scan lines in the observation region of the subject and extracts blood flow information using the acquired signals. In the ultrasonic diagnostic apparatus 1, the scan for generating a blood flow image may be performed by a system in which transmission/reception is performed a plurality of times on one scan line and then transmission/reception is performed on a next scan line or a system in which performing one transmission/reception on each scan line is repeated a plurality of times. In addition, in the generation of a B-mode image and the generation of a blood flow image, the ultrasonic diagnostic apparatus 1 can also transmit ultrasonic waves over a wide area of the observation region so as to reduce the number of scan line or, in other words, by transmitting plane waves or diffuse waves. Furthermore, the ultrasonic diagnostic apparatus 1 can also perform transmission/reception a plurality of times over a wide area of the observation region by changing a transmission angle of the plane wave or the diffuse wave and a range of the observation region to be transmitted, add the received signals, and use the added signals to generate an image.

In the present specification, both an analog signal output from the transducer 101 and digital data obtained by sampling (digitally converting) the output analog signal will be referred to as a received signal without particular distinction. However, a received signal may sometimes be referred to as received data depending on the context in order to clearly indicate that the received signal is digital data.

The received signal processing unit 106 is an image generating unit that generates image data based on a received signal obtained from the ultrasonic probe 102. The image processing unit 107 applies image processing such as brightness adjustment, interpolation, and filter processing on the image data generated by the received signal processing unit 106. The display unit 108 is a display unit that displays image data and various kinds of information related to images and is constituted by, for example, a liquid crystal display or an organic EL display. The system control unit 109 is a control unit that integrally controls the transmission electrical circuit 104, the reception electrical circuit 105, the received signal processing unit 106, the image processing unit 107, the display unit 108, and the like.

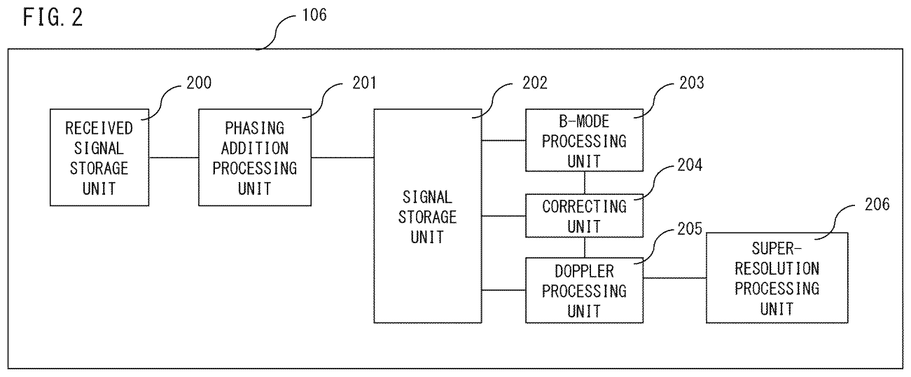

(Configuration of Received Signal Processing Unit) FIG. 2 is a block diagram showing an example of functions included in the received signal processing unit 106. As shown in FIG. 2, the received signal processing unit 106 includes a received signal storage unit 200, a phasing addition processing unit 201, a signal storage unit 202, a B-mode processing unit 203, a correcting unit 204, a Doppler processing unit 205, and a super-resolution processing unit 206.

The received signal storage unit 200 temporarily stores received signals received by the reception electrical circuit 105. Note that depending on an apparatus configuration of the ultrasonic diagnostic apparatus 1 and a type of the received signal, a configuration may be adopted in which the received signal is stored in the signal storage unit 202 provided in a stage subsequent to the phasing addition processing unit 201 to be described later instead of being stored in the received signal storage unit 200. In addition, the received signal storage unit 200 may be constituted of a common block with the signal storage unit 202 to be described later and may store the received signal from the reception electrical circuit 105 and a received signal after the phasing addition processing unit 201.

The phasing addition processing unit 201 performs phasing addition and quadrature detection processing on the received signal obtained by the reception electrical circuit 105 and stores the processed received signal in the signal storage unit 202. In this case, phasing addition processing refers to processing for forming a received ultrasonic beam by varying a delay time and a weight of signal addition for each transducer 101 and adding up received signals of the plurality of transducers 101 and is also called Delay and Sum (DAS) beamforming. In addition, quadrature detection processing refers to processing for converting a received signal into an in-phase signal (an I signal) and a quadrature signal (a Q signal) of a baseband. The phasing addition processing and the quadrature detection processing are performed on the basis of an element arrangement and various conditions of image generation (aperture control and signal filtering) that are input from the system control unit 109. The received signal after the phasing addition processing and the quadrature detection processing is stored in the signal storage unit 202. While typical DAS beamforming is assumed here, any processing that forms a received ultrasonic beam such as adaptive beamforming, model-based processing, or processing using machine learning may be adopted instead.

The B-mode processing unit 203 performs envelope detection processing, logarithmic compression processing, and the like on the received signal for B-mode image generation that is stored in the signal storage unit 202 and generates image data in which signal strength at each point inside of the observation region is expressed by brightness intensity. In addition, the B-mode processing unit 203 may perform B-mode processing with respect to a received signal after registering by the correcting unit 204 to be described later.

The correcting unit 204 calculates an amount of tissue displacement due to body motion and the like between frames with respect to the received signals of a plurality of frames. The correcting unit 204 calculates the amount of tissue displacement between frames by a block matching operation referred to as a speckle tracking method.

Specifically, the correcting unit 204 calculates an amount of displacement of a region of interest in a plurality of frames by setting the region of interest within a frame and tracking the region of interest between frames by correlation. In addition, the correcting unit 204 calculates an amount of displacement of an entire frame by setting a plurality of regions of interest in the frame. While an example of a method that uses a correlation between frames will be shown here, other methods may be adopted instead as long as the amount of displacement of a region of interest can be determined.

In the speckle tracking method, a speckle that is a scattered image of ultrasonic waves reflected from a scatterer in the tissue of the subject is tracked with respect to an ultrasonic signal. In the present embodiment, a calculation of the correlation of regions of interest between frames may be performed with respect to a received signal after any of phasing addition processing, quadrature detection processing, and envelope detection processing. In addition, the calculation of the correlation of regions of interest between frames may be performed with respect to a time waveform of the received signal or frequency space data of a discrete Fourier transform of the received signal. By moving the received signal with respect to a reference frame using the calculated amount of displacement, the correcting unit 204 registers tissues of the subject between frames. A movement amount of the received signal based on the amount of displacement may be a uniform movement amount for an entire frame using an average value of the amounts of displacement for each region of interest or may be a different movement amount for each region of interest in the frame. Furthermore, the amount of displacement calculated for each region of interest in the entire frame may be interpolated in a time direction of a data string within a frame and across a plurality of frames using linear interpolation, spline interpolation, or the like. In addition, the received signal may be moved after interpolating the received signal to be registered in a similar manner to the interpolation described above.

The Doppler processing unit 205 extracts blood flow information (Doppler information) from the received signal for blood flow image generation that is stored in the signal storage unit 202 and generates a blood flow image that visualizes the blood flow information. The Doppler processing unit 205 performs Doppler processing with respect to the received signal after movement by the correcting unit 204. In addition, the correcting unit 204 may calculate an amount of displacement between frames based on the blood flow image output by the Doppler processing unit 205 and perform registering processing. In the present embodiment, the correcting unit 204 may perform registering with respect to the blood flow image output from the Doppler processing unit 205 and output a signal after correction to the B-mode processing unit 203. In addition, after preforming a correction of the received signal for blood flow image generation stored in the signal storage unit 202, the correcting unit 204 may output, to the B-mode processing unit 203, a blood flow image obtained by having the Doppler processing unit 205 perform Doppler processing on the signal once again.

Processing by the Doppler processing unit 205 will now be described in detail. The Doppler processing unit 205 extracts blood flow information based on the Doppler effect of an object (in this case, blood) of the subject inside a scan range by performing a frequency analysis of a received signal for Doppler image generation that is stored in the signal storage unit 202. While a case where the object is blood is assumed in the present embodiment, alternatively, the object may be an object such as internal tissue or a contrast agent. In addition, an example of blood flow information includes at least any of a velocity, a dispersion value, and a power value of a blood flow. In this case, a power value is a value representing a strength of a blood flow signal indicated by a received signal and, as a specific example, a value obtained by squaring an absolute value of a received signal originating from a blood flow after reducing components such as those originating from stationary tissue or tissue with little movement. In the processing executed by the ultrasonic diagnostic apparatus 1 to be described hereinafter, the power value is assumed to be a power value obtained by the specific example described above. Furthermore, in this case, the components such as those originating from stationary tissue or tissue with little movement are clutter components related to the blood of the subject and are unwanted components other than signals from the blood flow.

Furthermore, the Doppler processing unit 205 may generate blood flow information at one point (one position) in the subject or generate blood flow information at a plurality of positions in a depth direction of the subject. In addition, the Doppler processing unit 205 may generate blood flow information at a plurality of time points in a time series. Accordingly, a time variation of the blood flow information can be displayed in the ultrasonic diagnostic apparatus 1.

In the generation of a blood flow image by the Doppler method according to the present embodiment, a received data string of a plurality of frames is acquired in a time direction at positions including the same position in the subject. The Doppler processing unit 205 applies a moving target indicator (MTI) filter with respect to the received data string. Accordingly, the Doppler processing unit 205 reduces clutter components and extracts components originating from the blood flow between frames. In addition, the Doppler processing unit 205 calculates blood flow information such as a velocity of blood flow, a dispersion of blood flow, and power of blood flow from the extracted component originating from the blood flow.

In addition, as the MTI filter used by the Doppler processing unit 205, a Butterworth-type infinite impulse response (IIR) filter may be adopted. Alternatively, as the MTI filter, a polynomial regression filter or the like may be adopted. Such filters with fixed filter coefficients can be adopted as the MTI filter. Alternatively, the MTI filter may be an adaptive filter that varies its coefficients according to an input signal using eigenvalue decomposition or singular value decomposition. In addition, the Doppler processing unit 205 can also decompose the received signal data into bases using eigenvalue decomposition, singular value decomposition, or the like and remove clutter components by extracting only specific bases.

The super-resolution processing unit 206 generates a super-resolution blood flow image that is a blood flow image with improved resolution from the blood flow image data generated by the Doppler processing unit 205. Details of processing of generating a super-resolution blood flow image according to the present embodiment will be described together with the following processing flow.

Image data output from the B-mode processing unit 203, the Doppler processing unit 205, and the super-resolution processing unit 206 is subjected to processing by the image processing unit 107 and finally displayed by the display unit 108. Each piece of image data may be displayed superimposed, displayed in parallel, or only some of the image data may be displayed.

The received signal processing unit 106 may be constituted by one or more processors and a memory. In this case, functions of the respective units of the received signal processing unit 106 shown in FIG. 2 are to be realized by a computer program. For example, the functions of the respective units of the received signal processing unit 106 can be provided by having a CPU load and execute a program stored in the memory. Other than the CPU, the received signal processing unit 106 may include a processor (a GPU, an FPGA, or the like) responsible for operations of the B-mode processing unit 203, the correcting unit 204, the Doppler processing unit 205, and the super-resolution processing unit 206. In addition, as the memory, a memory for storing a program in a non-transitory manner, a memory for temporarily saving data such as a received signal, or a working memory to be used by the CPU may be adopted.

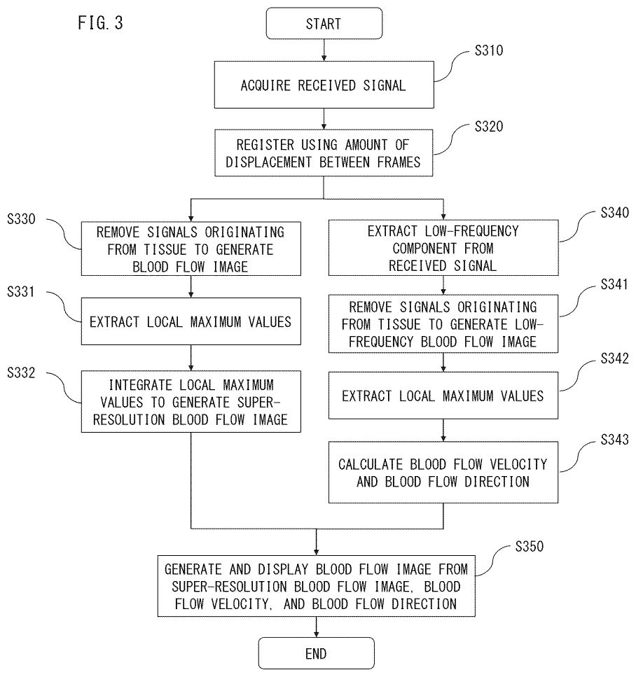

(Processing Flow of Blood Flow Image Generation) A configuration of the ultrasonic diagnostic apparatus 1 according to the first embodiment has been described above. Next, a processing flow for generating a blood flow image according to the present embodiment will be described. FIG. 3 shows a flow chart of processing of generating a super-resolution blood flow image according to the present embodiment. As an example, the system control unit 109 executes the processing of the present flowchart when transmission/reception of ultrasonic waves with respect to the subject 100 using the ultrasonic probe 102 starts in response to an operation of the ultrasonic diagnostic apparatus 1 by a user.

In step S310, the system control unit 109 repeatedly performs transmission/reception of ultrasonic waves by the ultrasonic probe 102 to the same position in an object region that is an object of acquisition of blood flow information in a plurality of frames. In addition, the system control unit 109 controls the reception electrical circuit 105 and generates a received signal including a data string of a plurality of consecutive frames in the time direction. The number of frames in a data string used to generate a super-resolution blood flow image is typically larger than the number of frames in a data string used to generate a normal Doppler image. For example, while the number of frames in a data string used to generate a normal Doppler image is about 5 to 20, the number of frames in a data string used to generate a super-resolution blood flow image is about several hundred to several tens of thousands. In this case, the system control unit 109 is a data acquiring unit that acquires data of reflected waves in the subject due to transmission/reception of ultrasonic waves over a plurality of frames.

In addition, the phasing addition processing unit 201 of the received signal processing unit 106 executes phasing addition processing and quadrature detection processing with respect to the generated received signal including the data string of a plurality of frames and stores the processed data in the signal storage unit 202. In the present embodiment, the data string for one frame may be a received signal obtained by performing one transmission/reception of ultrasonic waves on an object region or a signal obtained by adding up received signals obtained by performing transmission/reception of ultrasonic waves a plurality of times on the object region. For example, ultrasonic waves that are a plane wave or a diffuse wave can be transmitted over a range including the object region and a plurality of received signals received at different transmission angles can be added up to form a single frame's worth of data.

Next, in step S320, the correcting unit 204 calculates an amount of tissue displacement due to body motion and the like between frames from the received signal including the data string of a plurality of frames. In addition, by moving the received signal with respect to a reference frame using the calculated amount of displacement, the correcting unit 204 registers tissues of the subject between frames. At this point, as the reference frame when calculating the amount of displacement, one frame may be used for the entire data string, a plurality of reference frames may be used by changing the reference frame according to the position in the time direction, or a frame calculated on the basis of adjacent frames may be used.

Next, in step S330, the Doppler processing unit 205 generates a blood flow image from the received signal including the data string of a plurality of frames after the registering. As a specific example, the Doppler processing unit 205 applies an MTI filter to the received signal after the registering to reduce clutter components and extracts a component originating from the blood flow from the signal after filter application. In addition, the Doppler processing unit 205 generates blood flow images of a plurality of frames based on the extracted component originating from the blood flow. Note that in the present step, the MTI filter may be applied to a part of the data string after the registering or may be applied to the entire data string after the registering. The blood flow image generated in the present step is the fourth blood flow image indicating a blood flow of the subject that is generated using data with reduced clutter components.

Next, in step S331, the super-resolution processing unit 206 extracts local maximum values of power values in each frame. Note that the super-resolution processing unit 206 is not limited to extracting local maximum values and may extract maximum pixel values in each image or extract pixel values exceeding a predetermined threshold among maximum pixel values of the respective frames. In addition, the super-resolution processing unit 206 may extract pixel values in a predetermined range among a plurality of peaks of the pixel values in a localized region in each frame.

Next in step S332, the super-resolution processing unit 206 generates a super-resolution blood flow image by integrating local maximum values of the plurality of frames extracted in step S331. Since a position of the local maximum values moves between frames, the super-resolution processing unit 206 can generate a super-resolution blood flow image of which resolution has been improved over normal Doppler images by integrating a plurality of blood flow images. In the present step, the super-resolution processing unit 206 is the first image generating unit that generates the first blood flow image indicating a blood flow of the subject using data of a plurality of frames.

Next, in step S340, in order to calculate a blood flow velocity and a blood flow direction from the received signal of a plurality of frames, the Doppler processing unit 205 extracts a low-frequency component from the received signal including the data string of a plurality of frames after the registering. In this case, if the Doppler processing unit 205 can generate a low-frequency blood flow image from the extracted low-frequency signal, the Doppler processing unit 205 can use a filter with any frequency characteristics such as a low-pass filter to extract a low-frequency component from the data string. In addition, a frequency band to be an extraction object of the Doppler processing unit 205 may be set in advance according to sensitivity characteristics of the ultrasonic probe 102 or the user may designate any frequency band. For example, the display unit 108 may be a touch panel and configured as an accepting unit that accepts a designation of a blood flow velocity of the subject by the user. In addition, in the present step, the Doppler processing unit 205 is the second image generating unit that generates the second blood flow image including components with lower spatial frequency than the first blood flow image using data of a plurality of frames.

As will be described later, a frequency of the received signal suitable for calculating a blood flow velocity and a blood flow direction depends on the blood flow velocity to be calculated and a frame rate of the blood flow image. Therefore, in the present embodiment, the user may operate the ultrasonic diagnostic apparatus 1 to designate the blood flow velocity of the blood vessel that is an observation object and the Doppler processing unit 205 may determine a frame rate and a band to be used according to the designated blood flow velocity. In doing so, a movement distance of blood between frames is preferably equal to or less than half of a transmission wavelength of the transmitted ultrasonic waves. For example, let us assume a case where a blood vessel with a blood flow velocity of 100 m/s is an object of generation of a blood flow image under imaging conditions that include a center frequency of the transmitted ultrasonic waves being 10 MHz and a frame rate being 500 Hz. In this case, the movement distance of the blood between frames is 200 μm and a central frequency of the low-frequency region to be extracted is preferably set to 3 MHz or lower. In addition, in a case where a blood flow with a lower blood flow velocity than this example is an observation object, the frame rate needs to be set higher and the frequency band to be used needs to be set lower. Therefore, in this case, the system control unit 109 controls the transmission electrical circuit 104 so that a transmission frame rate thereof becomes a higher frame rate. In addition, the Doppler processing unit 205 changes filter settings so that lower frequencies are extracted.

Next, in step S341, the Doppler processing unit 205 applies an MTI filter to the received signal of the low-frequency component extracted in step S340 to reduce clutter components and extracts a component originating from the blood flow. In addition, the Doppler processing unit 205 generates low-frequency blood flow images of a plurality of frames using the extracted component originating from the blood flow. Subsequently, in step S342, the Doppler processing unit 205 extracts local maximum values of power values in each frame in a similar manner to step S331. In the present step, the Doppler processing unit 205 is the detection unit that detects maximum values of blood flow signals in a predetermined range of the second blood flow image.

Next, in step S343, the Doppler processing unit 205 calculates the blood flow velocity and the blood flow direction by tracking the local maximum values extracted in step S342 between frames. A general particle tracking algorithm can be used to track the local maximum values in the present step. For example, the Doppler processing unit 205 performs a nearest neighbor search using coordinates in a plane of the blood flow image for each local maximum between adjacent frames and calculates a movement distance and a movement direction of the local maximum before and after movement. In addition, the Doppler processing unit 205 calculates the blood flow velocity based on the calculated movement distance and a time interval between frames. In the present step, the Doppler processing unit 205 is the calculation unit that calculates the blood flow velocity and the blood flow direction of the subject based on positional information of detected maximum values.

In doing so, as a range of the nearest neighbor search, a range set in advance may be used or a range may be determined according to the object blood flow velocity. In addition, a tracking object need not be limited to adjacent frames and may be set to a plurality of (three or more) frames. Tracking may also be performed by a method such as pattern matching based on cross-correlation calculations using a plurality of particles instead of tracking a single particle in an image. When performing tracking of a particle, a mask intended to limit a search range to a blood flow region based on the power value of the blood flow image may be applied.

The Doppler processing unit 205 performs the tracking processing described above with respect to the received signals of a plurality of frames and calculates the blood flow velocity and the blood flow direction for each coordinate in the blood flow image. In this case, the blood flow velocity and the blood flow direction that are calculated for each local maximum varies from position to position and from frame to frame in the image due to the effects of blood flow velocity distribution in a blood vessel, peak tracking error, reconstructed pixel pitch, and the like. In consideration thereof, the blood flow velocity and the blood flow direction may be calculated for each pixel including a local maximum or numerical values based on a statistical index such as an average value, a mode, or a median of pixels including a local maximum in a predetermined region may be calculated as the blood flow velocity and the blood flow direction of the region. Alternatively, numerical values based on a statistical index such as an average value, a mode, or a median between frames may be calculated as the blood flow velocity and the blood flow direction of the region.

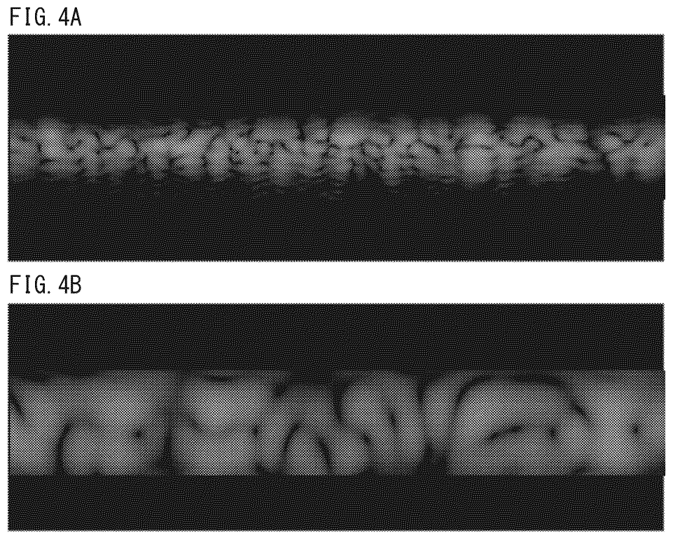

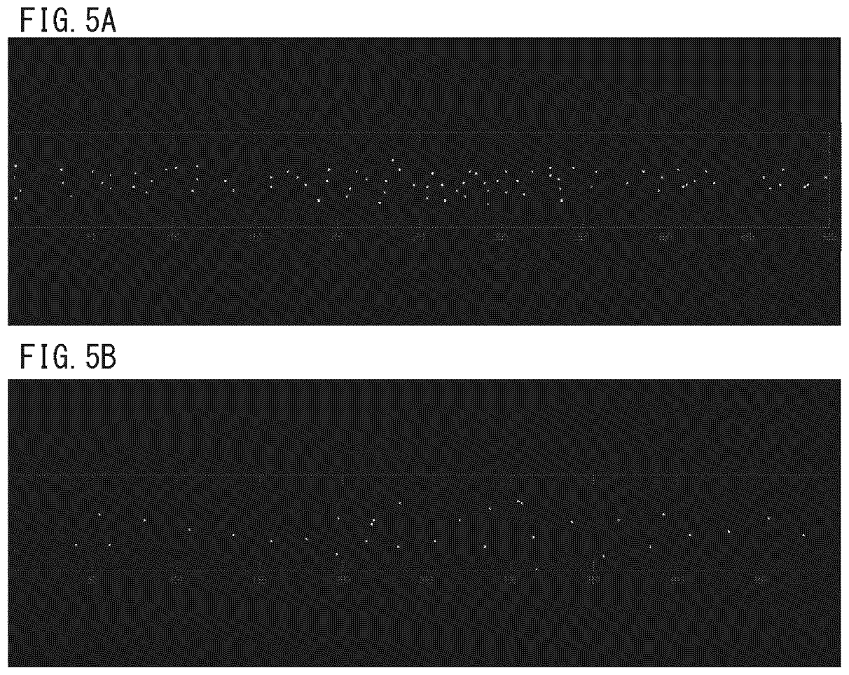

The reason for calculating a blood flow velocity and a blood flow direction from a low-frequency blood flow image will now be described. FIGS. 4A and 4B show examples of a blood flow image generated in step S330 and a low-frequency blood flow image generated in step S341. Compared to the blood flow image including a high-frequency component shown in FIG. 4A, the low-frequency blood flow image shown in FIG. 4B has a low spatial resolution and a rough speckle pattern. In addition, FIGS. 5A and 5B show examples of an image obtained by extracting local maximum values from a blood flow image including a high-frequency component generated in step S332 and an image obtained by extracting local maximum values from a low-frequency blood flow image generated in step S342. The images in FIGS. 5A and 5B show different densities of local maximum values depending on the respective spatial resolutions of the blood flow image and the low-frequency blood flow image in FIGS. 4A and 4B. In addition, as shown in FIGS. 5A and 5B, the density of the local maximum values of the blood flow image including the high-frequency component is higher than the density of the local maximum values of the low-frequency blood flow image.

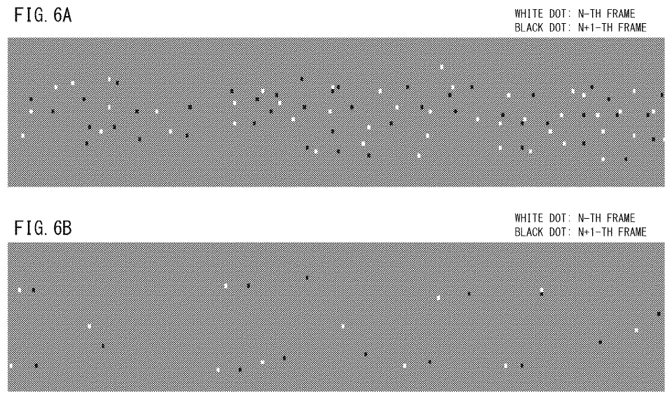

FIGS. 6A and 6B show an example of a movement of local maximum values between adjacent frames. In FIGS. 6A and 6B, local maximum values of an n-th frame are indicated by white dots and local maximum values of an n+1-th frame are indicated by black dots. In addition, it is assumed that the frame rates in the images in FIGS. 6A and 6B are the same. In this case, in a blood flow image including a high-frequency component, the density of local maximum values becomes higher than in a low-frequency blood flow image and a distance between a local maximum value of interest and the local maximum values present around the local maximum value of interest may become shorter than a movement distance between frames. As a result, a local maximum detected by a nearest neighbor search with respect to the local maximum value of interest may be another local maximum present around the local maximum value of interest instead of the local maximum value of interest itself after the movement. Therefore, in a blood flow image including a high-frequency component, the blood flow velocity and the blood flow direction may not be calculated correctly. On the other hand, in the low-frequency blood flow image shown in FIG. 6B, since the density of local maximum values is low, the local maximum value of interest is correctly tracked without being confused with the local maximum values surrounding the local maximum value of interest, making it more likely that the blood flow velocity and the blood flow direction for each pixel will be calculated correctly.

In addition, the accuracy of tracking local maximum values using blood flow images depends on the density of local maximum values in the blood flow image and the movement distance between frames. In consideration thereof, in the present embodiment, in the same blood flow image, a distance between adjacent local maximum values is desirably longer than the movement distance between frames. The density of local maximum values depends on a spatial frequency of a speckle pattern of the blood flow image. In addition, the movement distance between frames of local maximum values is determined by the blood flow velocity of the object blood flow and a frame rate of the blood flow image. In other words, in order to suitably calculate the blood flow velocity and the blood flow direction, desirably, the frame rate of the blood flow image is increased or the frequency of the received signal is lowered.

Here, a blood flow velocity v (x, y) at a position (x, y) on an xy-plane constituted of mutually orthogonal x and y axes in the blood flow image can be calculated by equation (1) below. In the equation, dx denotes a distance by which the local maximum values moves in an x-axis direction, dy denotes a distance by which the local maximum values moves in a y-axis direction, and dt denotes a time interval between frames.

$$\begin{matrix}[ Math . 1 ] & \\ v ( x , y ) = \sqrt{d {x}^{2} + d {y}^{2}} / dt & ( 1 )\end{matrix}$$

In addition, when a positive x-axis direction is 0 degrees, the blood flow direction (blood flow angle) θ (x, y) can be calculated by the following equation (2).

$$\begin{matrix}[ Math . 2 ] & \\ θ ( x , y ) = {tan}^{- 1} ( dy / dx ) & ( 2 )\end{matrix}$$

In addition, in step S350, the image processing unit 107 generates a super-resolution blood flow image indicating the blood flow velocity and the blood flow direction using the super-resolution blood flow image generated in step S332 and the blood flow velocity and the blood flow image of each pixel calculated in step S343. In the present step, the image processing unit 107 is the third image generating unit that generates the third blood flow image based on the first blood flow image, the blood flow velocity, and the blood flow direction. In addition, in the present step, the image processing unit 107 colors the blood flow velocity and the blood flow direction in the generated super-resolution blood flow image, respectively. For example, the image processing unit 107 selects pixels having a power value equal to or greater than a specific threshold from the super-resolution blood flow image and assigns a hue in accordance with the blood flow velocity and the blood flow direction calculated in step S343 with respect to the selected pixels. In addition, the super-resolution blood flow image generated by the image processing unit 107 is displayed on the display unit 108. Note that as the display of the super-resolution blood flow image on the display unit 108, the B-mode image, the super-resolution blood flow image, the blood flow velocity, and the blood flow direction may be displayed in any combination in parallel or the B-mode image, the super-resolution blood flow image, the blood flow velocity, and the blood flow direction may be switched and displayed. In addition, the image processing unit 107 may further generate a histogram indicating variability of the blood flow velocity and the blood flow direction and the generated histogram may be displayed on the display unit 108. Furthermore, the image processing unit 107 may generate the super-resolution blood flow image, the blood flow velocity, and the blood flow direction (vector indicating blood flow direction) as the third blood flow image described above and the generated blood flow image may be displayed in parallel and/or in an overlapping manner on the display unit 108.

In addition, the image processing unit 107 can generate a transformed image converted to RGB by a two-dimensional color map of the blood flow velocity in a super-resolution image and the power value in a super-resolution blood flow image. For example, the image processing unit 107 can generate the transformed image using a two-dimensional color map in which the blood flow velocity is assigned to hue and the power value of the super-resolution blood flow image is assigned to lightness. In addition, the image processing unit 107 can also determine hues based on a circular gradation map that changes the hue in the blood flow direction into a rotation direction on the xy plane. Furthermore, the image processing unit 107 may determine the hue in the blood flow direction using a color map that varies in one dimension with respect to any direction on the xy plane. For example, the image processing unit 107 can indicate a difference in blood flow directions by hue by representing a rightward direction of the screen (positive x-axis direction) in red colors and a leftward direction (negative x-axis direction) in blue colors. In addition, the image processing unit 107 may determine the hue based on a two-dimensional color map corresponding to a flow velocity vector determined by the blood flow velocity and the blood flow direction. For example, the image processing unit 107 can use a two-dimensional color map in which the blood flow direction is assigned to hue and the blood flow velocity is assigned to lightness.

As described above, the ultrasonic diagnostic apparatus 1 according to the present embodiment can generate a super-resolution blood flow image representing a blood flow velocity and a blood flow direction that are calculated more accurately than conventional techniques using a low-frequency component of a received signal obtained by transmission/reception of ultrasonic waves with respect to a subject.

While the ultrasonic diagnostic apparatus 1 executes the processing of steps S330 to S332 and the processing of steps S340 to S343 in parallel in the present embodiment, the processing of steps S330 to S332 and the processing of steps S340 to S343 may be executed separately, respectively.

### Second Embodiment

Next, an ultrasonic diagnostic apparatus according to a second embodiment will be described. It should be noted that, in the following description, components and processing similar to those of the ultrasonic diagnostic apparatus 1 according to the first embodiment will be denoted by the same reference signs and detailed descriptions thereof will be omitted.

The ultrasonic diagnostic apparatus 1 according to the first embodiment generates a super-resolution blood flow image by coloring a blood flow velocity and a blood flow direction for each position of local maximum values calculated from a low-frequency blood flow image. On the other hand, the ultrasonic diagnostic apparatus 1 according to the second embodiment displays the blood flow velocity and the blood flow direction by integrating and drawing trajectories of local maximum values between frames.

The ultrasonic diagnostic apparatus 1 according to the present embodiment executes processing of the flow chart shown in FIG. 3. However, in step S343 of the present embodiment, a blood flow velocity and a blood flow direction are calculated by processing that differs from the first embodiment. In the first embodiment, the Doppler processing unit 205 calculates the blood flow velocity and the blood flow direction using coordinates of local maximum values for each frame. On the other hand, in the present embodiment, the Doppler processing unit 205 calculates the blood flow velocity and the blood flow direction by integrating trajectories that connect coordinates of the local maximum values before and after movement between frames by straight lines over a plurality of frames. By integrating trajectories and calculating the blood flow velocity and the blood flow direction in this manner, the ultrasonic diagnostic apparatus 1 according to the present embodiment can calculate the blood flow velocity and the blood flow direction of a blood vessel using a smaller number of frames than when integrating coordinates as in the first embodiment.

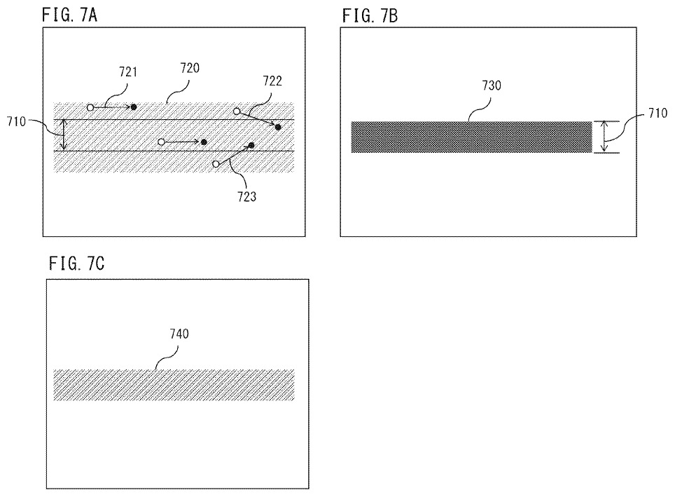

However, as shown in FIGS. 4A and 4B, since a low-frequency blood flow image has low spatial resolution, the spatial resolution of an image showing a blood flow velocity generated by integrating trajectories of local maximum values of a low-frequency blood flow image in the present embodiment will also be low. FIG. 7A shows a low-frequency blood flow image generated by the ultrasonic diagnostic apparatus 1 in the present embodiment. Since a low-frequency blood flow image has low spatial resolution, local maximum values may occur outside of blood vessels. Therefore, as shown in FIG. 7A, when trajectories 721, 722, and 723 of local maximum values outside of blood vessels are integrated, a blood vessel 720 in the low-frequency blood flow image is drawn thicker than an actual blood vessel diameter 710 of the blood vessel.

FIG. 7B shows a super-resolution blood flow image 730 generated in step S332 in the present embodiment. As shown in FIG. 7B, a super-resolution blood flow image generated from a received signal including a high-frequency component has a higher spatial resolution than a low-frequency blood flow image and a blood vessel diameter drawn in the super-resolution blood flow image will theoretically be the same as the actual blood vessel diameter 710 of the blood vessel. In the ultrasonic diagnostic apparatus 1 according to the present embodiment, a super-resolution blood flow image in FIG. 7B is used as a mask image on a low-frequency blood flow image in FIG. 7A to generate a super-resolution blood flow image in FIG. 7C.

As a specific example, the image processing unit 107 performs threshold processing based on the power values of the super-resolution blood flow image to generate a binary image in which a value of “0” or “1” is set for each pixel. For example, the image processing unit 107 sets a pixel value of “0” to pixels of which the power value is lower than a threshold and sets a pixel value of “1” to pixels of which the power value is equal to or higher than the threshold. By applying the generated binary image to the low-frequency blood flow image, the image processing unit 107 uses the binary image as a mask image to exclude the blood flow velocity and the blood flow direction outside of a vascular region and to extract a vascular structure. The image processing unit 107 generates a super-resolution blood flow image 740 shown in FIG. 7C by assigning transparency corresponding to pixel values of “0” or “1” and superimposing and displaying the mask image with the low-frequency blood flow image. Note that the mask image used in the present step need not be a binary image and, for example, power values of a super-resolution blood flow image may be used as pixel values. Alternatively, by assigning a transparency to pixel values of the mask image and further increasing the transparency of pixels with large power values, the image processing unit 107 can generate a blood flow image with a further enhanced vascular structure.

As described above, with the ultrasonic diagnostic apparatus 1 according to the present embodiment, a user can more accurately comprehend the blood flow velocity and the blood flow direction of a blood vessel by generating a blood flow image in which blood vessels of a subject are drawn with higher accuracy.

### Third Embodiment

Next, an ultrasonic diagnostic apparatus according to a third embodiment will be described. It should be noted that, in the following description, components and processing similar to those of the ultrasonic diagnostic apparatus 1 according to the first embodiment or the second embodiment will be denoted by the same reference signs and detailed descriptions thereof will be omitted.

In the ultrasonic diagnostic apparatus 1 according to the first embodiment, in order to calculate a blood flow velocity and a blood flow direction, a blood flow image is generated by extracting a low-frequency component from a received signal including a data string of a plurality of frames in a time direction with a band filter (steps S340 and S341). On the other hand, the ultrasonic diagnostic apparatus 1 according to the present embodiment generates a low-frequency blood flow image using data acquired by transmission/reception of ultrasonic waves that differs from transmission/reception of ultrasonic waves for acquiring data to be used to generate a high-frequency blood flow image. In this manner, received signals acquired by transmission/reception of different ultrasonic waves are used to generate the respective blood flow images. Accordingly, a difference between a low-frequency blood flow image and a high-frequency blood flow image can be increased as compared to a case of extracting a low-frequency component from the same received signal as the received signal used to generate the high-frequency blood flow image. As a result, in the ultrasonic diagnostic apparatus 1 according to the present embodiment, a blood flow velocity and a blood flow direction can be calculated faster and a super-resolution blood flow image with higher resolution can be generated.

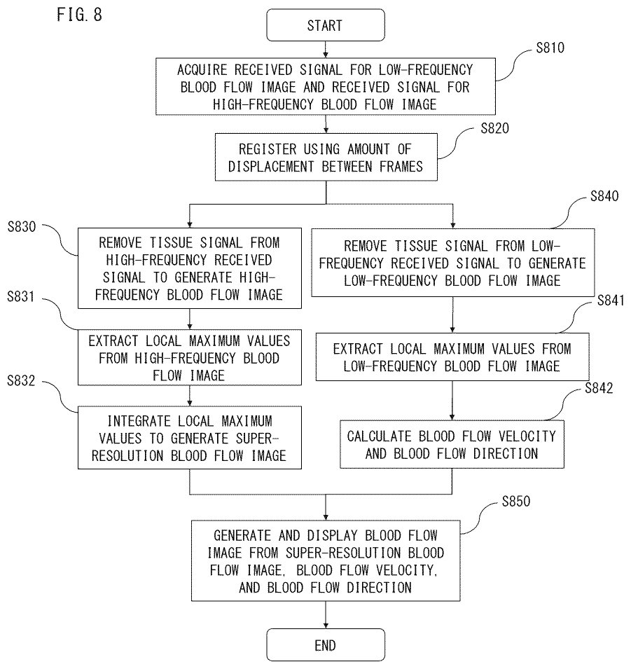

FIG. 8 shows an example of a flow chart of processing executed by the ultrasonic diagnostic apparatus 1 according to the present embodiment. In step S810, by the ultrasonic probe 102, the system control unit 109 repeatedly performs respective transmission/reception of ultrasonic waves for generating a low-frequency blood flow image and a high-frequency blood flow image with respect to the same position in an object region that is an object of acquisition of blood flow information in a plurality of frames. Accordingly, the system control unit 109 acquires a low-frequency received signal and a high-frequency received signal including a data string of a plurality of frames in the time direction. Note that in the present step, the system control unit 109 may alternate low-frequency transmissions and high-frequency transmissions by the ultrasonic probe 102 one wave at a time or a plurality of transmissions at a time. In addition, when a body motion of the subject is large and there is a possibility of positional deviation in a short period of time, the system control unit 109 may alternate between low-frequency transmissions and high-frequency transmissions with the ultrasonic probe 102 using the fewest number of waves possible.

Next, in step S820, the correcting unit 204 calculates an amount of tissue displacement due to body motion and the like between frames from the received signal including the data string of a plurality of frames in a similar manner to step S320. In addition, by moving the received signal with respect to a reference frame using the calculated amount of displacement, the correcting unit 204 registers tissues of the subject between frames. In the present step, the correcting unit 204 may perform the processing of registering independently for a low-frequency image and a high-frequency image, respectively, or perform registering in the low-frequency image based on the results of registering in the high-frequency image with a higher spatial resolution.

Next, in step S830, the super-resolution processing unit 206 generates a high-frequency blood flow image from a high-frequency received signal including the data string of a plurality of frames after the registering. In addition, in step S840, the Doppler processing unit 205 generates a low-frequency blood flow image from a low-frequency received signal including the data string of a plurality of frames after the registering. Since the steps S831, S832, S841, S842, and S850 are the same processing as the steps S331, S332, S342, S343, and S350, respectively, a detailed description will be omitted here.

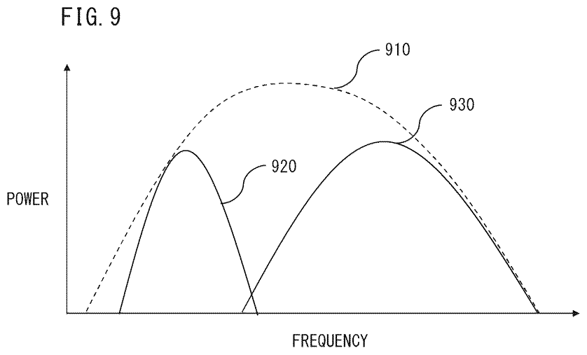

FIG. 9 is a graph showing an example of a frequency band of a received signal acquired by transmission/reception of ultrasonic waves in the present embodiment. In FIG. 9, an abscissa represents a frequency of a received signal and an ordinate represents a power value of the received signal. The ultrasonic diagnostic apparatus 1 according to the present embodiment acquires received signals of a low-frequency band 920 and a high-frequency band 930 by respectively different transmission/reception and band filter processing in a frequency band (dashed line: 910) that the ultrasonic probe 102 can transmit/receive. In addition, in the ultrasonic diagnostic apparatus 1, fractional bandwidths of the high-frequency band 930 and the low-frequency band 920 are preferably set about the same in order to prevent the generation of unwanted artifacts in the generated blood flow image.

While the ultrasonic diagnostic apparatus 1 executes the processing of steps S830 to S832 and the processing of steps S840 to S842 in parallel in the present embodiment, the processing of steps S830 to S832 and the processing of steps S840 to S842 may be executed separately, respectively.

### Fourth Embodiment

Next, an ultrasonic diagnostic apparatus according to a fourth embodiment will be described. It should be noted that, in the following description, components and processing similar to those of the ultrasonic diagnostic apparatus 1 according to the first embodiment to the third embodiment will be denoted by the same reference signs and detailed descriptions thereof will be omitted.

In the first embodiment, the second embodiments and the third embodiment, a case of generating a super-resolution blood flow image showing a blood flow velocity and a blood flow direction of a two-dimensional image is assumed. On the other hand, in the ultrasonic diagnostic apparatus 1 according to the present embodiment, a super-resolution blood flow image low image showing a three-dimensional blood flow velocity and a three-dimensional blood flow direction is generated.

In the present embodiment, using the ultrasonic probe 102 that is a two-dimensional probe, transmission/reception of ultrasonic waves are repeated at the same position to include an object region that is an object of acquisition of blood flow information. In the present embodiment, the ultrasonic diagnostic apparatus 1 executes processing of the flow chart shown in FIG. 3 or FIG. 8. A detailed description of the processing of each step will not be provided here.

The received signal processing unit 106 generates a received signal including a data string of a plurality of frames in the time direction. In addition, the Doppler processing unit 205 generates three-dimensional blood flow data using the acquired received signal and extracts three-dimensional local maximum values in a plurality of frames. Furthermore, the super-resolution processing unit 206 generates three-dimensional super-resolution blood flow data by integrating the three-dimensional local maximum values. In addition, the Doppler processing unit 205 generates three-dimensional low-frequency blood flow data using a low-frequency component of the received signal and generates three-dimensional low-frequency local maximum data. Furthermore, the Doppler processing unit 205 calculates a three-dimensional blood flow velocity and a three-dimensional blood flow direction by tracking low-frequency local maximum values using three-dimensional coordinates between frames. In addition, the image processing unit 107 performs rendering processing with respect to the three-dimensional super-resolution blood flow data and the data of the three-dimensional blood flow velocity and the three-dimensional blood flow direction and generates various kinds of two-dimensional image data to be displayed on the display unit 108.

Therefore, by applying the processing described above to the processing of the flow chart shown in FIG. 3 or FIG. 8, the ultrasonic diagnostic apparatus 1 according to the present embodiment can generate a super-resolution blood flow image showing a three-dimensional blood flow velocity and a three-dimensional blood flow direction.

The technique according to the present disclosure can improve accuracy of blood flow images.

Note that the above-described various types of control may be processing that is carried out by one piece of hardware (e.g., processor or circuit), or otherwise. Processing may be shared among a plurality of pieces of hardware (e.g., a plurality of processors, a plurality of circuits, or a combination of one or more processors and one or more circuits), thereby carrying out the control of the entire device.

Also, the above processor is a processor in the broad sense, and includes general-purpose processors and dedicated processors. Examples of general-purpose processors include a central processing unit (CPU), a micro processing unit (MPU), a digital signal processor (DSP), and so forth. Examples of dedicated processors include a graphics processing unit (GPU), an application-specific integrated circuit (ASIC), a programmable logic device (PLD), and so forth. Examples of PLDs include a field-programmable gate array (FPGA), a complex programmable logic device (CPLD), and so forth.

The embodiment described above (including variation examples) is merely an example. Any configurations obtained by suitably modifying or changing some configurations of the embodiment within the scope of the subject matter of the present disclosure are also included in the present disclosure. The present disclosure also includes other configurations obtained by suitably combining various features of the embodiment.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-153377, filed on Sep. 5, 2024, which is hereby incorporated by reference herein in its entirety.

What is claimed is:

1. An ultrasonic diagnostic apparatus, comprising:
a processor; and
a memory storing a program which, when executed by the processor, causes the ultrasonic diagnostic apparatus to
execute ultrasonic wave transmission and reception processing of transmitting and receiving ultrasonic waves with respect to a subject,
execute data acquisition processing of acquiring data of reflected waves in the subject due to the transmission and reception of ultrasonic waves over a plurality of frames,
execute first image generation processing of generating a first blood flow image indicating a blood flow of the subject using the data of the plurality of frames acquired by the data acquisition processing,
execute second image generation processing of generating a second blood flow image including a component with a lower spatial frequency than the first blood flow image using the data of the plurality of frames acquired by the data acquisition processing,
execute detection processing of detecting maximum values of blood flow signals in a predetermined range of the second blood flow image,
execute calculation processing of calculating a blood flow velocity and a blood flow direction of the subject based on positional information of the maximum values detected by the detection processing, and
execute third image generation processing of generating a third blood flow image based on the first blood flow image, the blood flow velocity, and the blood flow direction.

2. The ultrasonic diagnostic apparatus according to claim 1, wherein
in the first image generation processing,
clutter components related to a blood flow of the subject included in the data of the plurality of frames acquired by the data acquisition processing are reduced,
a fourth blood flow image indicating a blood flow of the subject is generated using data with the reduced clutter components,
maximum values of blood flow signals in a predetermined range of the fourth blood flow image are detected, and
the first blood flow image is generated by integrating the detected maximum values.

3. The ultrasonic diagnostic apparatus according to claim 1, wherein in the second image generation processing, the second blood flow image is generated using a low-frequency component of the data of the plurality of frames acquired by the data acquisition processing.

4. The ultrasonic diagnostic apparatus according to claim 1, wherein in the second image generation processing, the second blood flow image is generated using the data acquired by the data acquisition processing by transmission and reception of the ultrasonic waves which differs from the transmission and reception of the ultrasonic wave for acquiring the data used to generate the first blood flow image.

5. The ultrasonic diagnostic apparatus according to claim 1, wherein in the data acquisition processing, an amount of displacement of tissue of the subject included in the data of the reflected waves is calculated, and data obtained by performing registering of the tissue with respect to data of the reflected waves using the calculated amount of displacement is acquired as the data of the plurality of frames.

6. The ultrasonic diagnostic apparatus according to claim 1, wherein
the program, when executed by the processor, further causes the ultrasonic diagnostic apparatus to execute acceptance processing of accepting a designation of a flow velocity of a blood flow of the subject by a user, and
in the second image generation processing, the second blood flow image is generated based on a frame rate and a band determined according to the flow velocity accepted by the acceptance processing.

7. The ultrasonic diagnostic apparatus according to claim 6, wherein in the second image generation processing, the frame rate is determined so that a movement distance of blood between frames indicated by the flow velocity is equal to or less than half of a transmission wavelength of the ultrasonic waves.

8. The ultrasonic diagnostic apparatus according to claim 1, wherein in the third image generation processing, the third blood flow image is generated by coloring the blood flow velocity and the blood flow direction according to a position of the maximum values detected by the detection processing.

9. The ultrasonic diagnostic apparatus according to claim 1, wherein in the third image generation processing, the third blood flow image is generated by integrating trajectories connecting the maximum values detected by the detection processing over the plurality of frames.

10. The ultrasonic diagnostic apparatus according to claim 1, wherein in the third image generation processing, the third blood flow image is generated by excluding the blood flow velocity and the blood flow direction outside of a vascular region indicated by the first blood flow image.

11. A control method of an ultrasonic diagnostic apparatus, comprising:
transmitting and receiving ultrasonic waves with respect to a subject;
acquiring data of reflected waves in the subject due to transmission and reception of the ultrasonic waves over a plurality of frames;
generating a first blood flow image indicating a blood flow of the subject using the data of the plurality of frames acquired by the acquiring of data;
generating a second blood flow image including a component with a lower spatial frequency than the first blood flow image using the data of the plurality of frames acquired by the acquiring of data;
detecting maximum values of blood flow signals in a predetermined range of the second blood flow image;
calculating a blood flow velocity and a blood flow direction of the subject based on positional information of the maximum values detected by the detecting of a maximum values; and
generating a third blood flow image based on the first blood flow image, the blood flow velocity, and the blood flow direction.

12. A non-transitory computer readable medium that stores a program, wherein the program causes a computer to execute a control method of an ultrasonic diagnostic apparatus, the control method comprising:
transmitting and receiving ultrasonic waves with respect to a subject;
acquiring data of reflected waves in the subject due to transmission and reception of the ultrasonic waves over a plurality of frames;
generating a first blood flow image indicating a blood flow of the subject using the data of the plurality of frames acquired by the acquiring of data;
generating a second blood flow image including a component with a lower spatial frequency than the first blood flow image using the data of the plurality of frames acquired by the acquiring of data;
detecting maximum values of blood flow signals in a predetermined range of the second blood flow image;
calculating a blood flow velocity and a blood flow direction of the subject based on positional information of the maximum values detected by the detecting of a maximum values; and
generating a third blood flow image based on the first blood flow image, the blood flow velocity, and the blood flow direction.
